(19) United States
(12) Patent Application Publication
(10) Pub. No.: US 2024/0358673 A1
(43) Pub. Date: Oct. 31, 2024

(54) SHORT CHAIN FATTY ACIDS & GALLATES COMPOSITIONS

(71) Applicant: Franco Cavaleri, Surrey (CA)

(72) Inventor: Franco Cavaleri, Surrey (CA)

(21) Appl. No.: 18/377,582

(22) Filed: Oct. 6, 2023

Related U.S. Application Data
(60) Provisional application No. 63/462,145, filed on Apr. 26, 2023.

(51) Int. Cl. A61K 31/352; A61K 9/48; A61K 31/19; A61P 3/04

(52) U.S. Cl. CPC A61K 31/352; A61K 9/4816; A61K 31/19; A61P 3/04

(57) ABSTRACT

Compositions and methods for providing a person with an exogenous and therapeutically effective supply of short chain fatty acids, gallates, and (optionally) one or more ketones are disclosed. The compositions include one or more short chain fatty acids (such as butyric acid, butyrate salts, propionic acid, propionate salts, acetic acid, or acetate salts); epigallocatechin gallate (EGCG); and, optionally, one or more beta-hydroxybutyrate salts. The compositions and methods are useful for inducing weight loss, ketosis, brown adipose fat cell differentiation, and/or beta-oxidative activity in a subject.

### CROSS-REFERENCE TO RELATED APPLICATIONS

This application claims priority to, and incorporates by reference, U.S. provisional patent application Ser. No. 63/462,145, filed on Apr. 26, 2023.

### FIELD OF THE INVENTION

The field of the present invention relates to certain compositions (and methods of use thereof) that comprise a combination of short chain fatty acids (such as butyrate); one or more gallates (such as epigallocatechin gallate (EGCG)); and, optionally, one or more ketones (such as beta-hydroxybutyrate), which provide the various health benefits described herein.

### BACKGROUND OF THE INVENTION

There is an ongoing need within society for dietary and nutritional supplements that are effective to reduce inflammation, assist with weight loss, and to help prevent heart disease. Although there are currently-available pharmaceutical products that are specifically designed to address certain of those conditions, there is a continuing need for less expensive dietary and nutritional supplements that may be used and consumed to address the same needs.

As the following will demonstrate, the compositions and methods described herein are useful for providing an exogenous supply of short chain fatty acids, gallates, and (optionally) one or more ketones, which have been shown to provide a person with an effective means to reduce inflammation, assist with weight loss, and to help prevent heart disease.

### SUMMARY OF THE INVENTION

According to certain aspects of the invention, compositions are provided that include combinations of short chain fatty acids (e.g., butyric acid, butyrate salts, propionic acid, propionate salts, acetic acid, or acetate salts); one or more gallates (e.g., epigallocatechin gallate (EGCG)); and, optionally, one or more ketones (particularly beta-hydroxybutyrate), and/or derivatives of the foregoing. According to certain preferred aspects of the invention, the one or more gallates include or, in some embodiments, consist essentially of epigallocatechin gallate (EGCG). The compositions of the present invention offer a multitude of benefits and can be used for numerous applications. For example, oral formulations of such compositions may be used to provide a person with an effective means to reduce inflammation, to assist with weight loss, to help prevent heart disease, to activate uncoupling protein 1 (UCP1), to induce thermogenic activity, to induce lipolysis, to induce brown adipose fat cell differentiation, and/or to modulate Sirtuin protein expression.

The above-mentioned and additional features of the present invention are further illustrated in the Detailed Description contained herein.

### BRIEF DESCRIPTION OF THE FIGURES

FIG. 1: two histology slides showing the effects of a composition of the present invention on brown fat cell differentiation and activation.

FIG. 2: a bar graph that summarizes the effects of a composition of the present invention on Sirtuin 1 expression.

FIG. 3: a table summarizing the results of a human weight loss study involving a composition of the present invention.

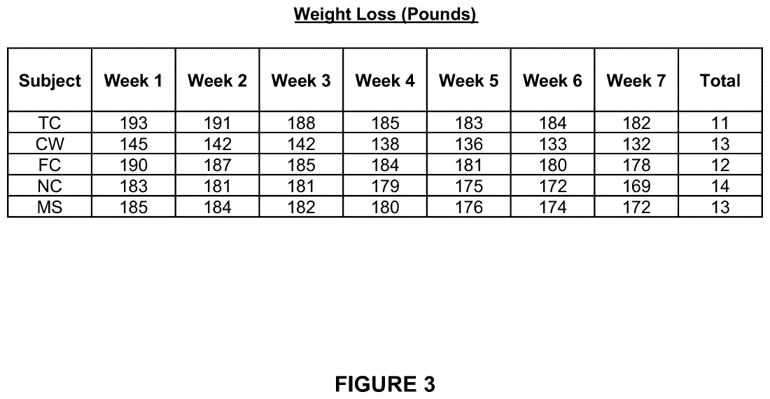

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used, and made without departing from the scope and spirit of the invention.

According to certain preferred embodiments of the present invention, compositions are provided that include a combination of short chain fatty acids (such as butyric acid, butyrate salts, propionic acid, propionate salts, acetic acid, or acetate salts); one or more gallates; and, optionally, one or more ketones (e.g., beta-hydroxybutyrate), and/or derivatives of the foregoing. In certain preferred embodiments of the invention, the one or more gallates include, consist essentially of, or consist entirely of purified epigallocatechin gallate, (2R,3R)-3′, 4′, 5,5′, 7-Pentahydroxyflavan-3-yl 3,4,5-trihydroxybenzoate, also known as epigallocatechin-3-gallate, the structure of which is reproduced below.

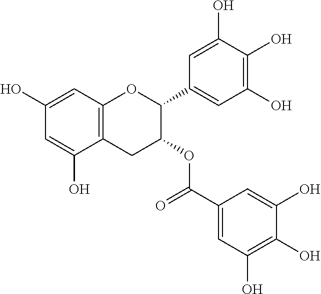 [SMILES: O=C(O[C@@H]1Cc2c(O)cc(O)cc2O[C@@H]1c1cc(O)c(O)c(O)c1)c1cc(O)c(O)c(O)c1]

In certain preferred embodiments, the short chain fatty acids consist of purified butyric acid, butyrate salts, propionic acid, propionate salts, acetic acid, and/or acetate salts. In certain preferred embodiments, the short chain fatty acids consist of purified butyrate salts, including butyrate sodium salt, butyrate calcium salt, and/or butyrate magnesium salt. As used herein, the one or more gallates included in the compositions include any salt or ester of gallic acid. In certain preferred embodiments, the one or more gallates include, consists essentially of, or consists entirely of, purified epigallocatechin gallate (EGCG). In such embodiments, the inclusion of elevated amounts of short chain fatty acids (e.g., butyrate) and epigallocatechin gallate (EGCG) in the compositions of the invention will provide a therapeutic source of short chain fatty acids that is effective to induce ketosis in a subject—and, furthermore, it will supply a significant amount of EGCG for optimal antioxidant support.

Still further, the invention provides that the compositions of the present invention encompass EGCG and one or more sources of short chain fatty acids (such as butyric acid or butyrate salts) as separate, but mixed, chemical components; whereas, in other embodiments, the EGCG and one or more sources of short chain fatty acids (such as butyric acid or butyrate salts) may be covalently bonded to each other (either directly or through an intermediary linker). In addition, the invention provides that the EGCG may exist in a natural extract, such as green tea extract, or the EGCG may exist in a purified form, such as at least 75%, 80%, 85%, 90%, or 95% purified (i.e., free from other compositions). The invention provides that the one or more sources of short chain fatty acids (such as butyric acid, butyrate salts, propionic acid, propionate salts, acetic acid, or acetate salts) will exist in a purified form, such as at least 75%, 80%, 85%, 90%, or 95% purified (i.e., free from other compositions).

The invention provides that the compositions may further include other pharmacologically active agents, such as acetyl-L-carnitine, R-alpha lipoic acid, green tea extract, vitamins, and various combinations of such agents. According to additional embodiments of the present invention, the compositions may (optionally) further include one or more ketones (or precursors thereof), such as purified beta-hydroxybutyrate or esters or propionate salts thereof. Non-limiting examples of such purified beta-hydroxybutyrate salts include i a beta-hydroxybutyrate sodium salt, a beta-hydroxybutyrate calcium salt, and/or a beta-hydroxybutyrate magnesium salt. The invention provides that the one or more sources of ketones (such as purified beta-hydroxybutyrate or esters or propionate salts thereof) will exist in a purified form, such as at least 75%, 80%, 85%, 90%, or 95% purified (i.e., free from other compositions).

In certain embodiments, the present invention further includes methods for reducing inflammation, assisting with weight loss, and/or helping to prevent heart disease. Still further, the present invention includes methods for activating uncoupling protein 1 (UCP1), inducing thermogenic activity, inducing lipolysis, and/or to inducing brown adipose fat cell differentiation in a subject. According to additional embodiments, the present invention includes methods of using the compositions described herein to modulate Sirtuin protein expression. Sirtuin proteins represent a group of subcellular proteins that, when expressed, are believed to reverse age- and metabolic-related syndromes. Sirtuin proteins include SIRT1, SIRT6, and SIRT7 (which are predominately located in the nucleus of a cell); SIRT2 (primarily located in cytoplasm); and SIRT3, SIRT4, and SIRT5 (primarily located in mitochondria). Such methods generally include orally administering one of the pharmacologic compositions described herein to a subject, thereby indirectly applying such compositions to cells that can then express sirtuin proteins.

In such embodiments, the methods and compositions are effective to deliver at least 100 mg of one or more gallates (such as epigallocatechin gallate (EGCG)) and at least 50 mg of short chain fatty acids (such as butyric acid, butyrate salts, propionic acid, propionate salts, acetic acid, or acetate salts). Such amounts may be administered within a single dose or, alternatively, through a multiple dose/daily regimen. In other embodiments, the methods and compositions are effective to deliver at least 100 mg, 200 mg, 300 mg, 400 mg, 500 mg, 600 mg, 700 mg, 800 mg, or 900 mg of short chain fatty acid (such as butyrate salts) and at least 200 mg, 300 mg, 400 mg, 500 mg, 600 mg, 700 mg, 800 mg, or 900 mg of one or more gallates (such as epigallocatechin gallate (EGCG)). In still further embodiments, the methods and compositions may optionally further be effective to deliver at least 100 mg, 200 mg, 300 mg, 400 mg, 500 mg, 600 mg, 700 mg, 800 mg, or 900 mg of ketones (such as a beta-hydroxybutyrate sodium salt, a beta-hydroxybutyrate calcium salt, and/or a beta-hydroxybutyrate magnesium salt). As described further below in the Examples, such compositions are preferably delivered to a person in the form of oral capsules or dry powders.

Notwithstanding the preferred embodiments and Examples described herein, the invention provides that the compositions of the present invention may be administered in any desired and effective manner, e.g., as pharmaceutical compositions or nutritional supplements for oral ingestion. More particularly, for example, pharmaceutically acceptable compositions or nutritional supplements of the invention may comprise one or more of the compositions described herein with one or more acceptable carriers. Regardless of the route of administration selected, the compositions may be formulated into acceptable dosage forms by conventional methods known to those of skill in the art. For example, acceptable carriers include, but are not limited to, sugars (e.g., lactose, sucrose, mannitol, and sorbitol), silicon dioxide, starches, cellulose preparations (such as microcrystalline cellulose), calcium phosphates (e.g., dicalcium phosphate, tricalcium phosphate and calcium hydrogen phosphate), sodium citrate, water, aqueous solutions, alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly (orthoesters), and poly (anhydrides)), elastomeric matrices, liposomes, microspheres, oils (e.g., corn, germ, olive, castor, sesame, cottonseed, and groundnut), cocoa butter, waxes, paraffins, silicones, talc, silicylate, etc.

Each acceptable carrier used in a pharmaceutical composition or nutritional supplement of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions and nutritional supplements of the invention may, optionally, contain additional ingredients and/or materials commonly used in pharmaceutical compositions and/or nutritional supplements. These ingredients and materials include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxy methyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; (28) vitamins and minerals; (29) proteins that carry therapeutic or nutritional benefits, such as whey protein and other milk-derived proteins; and (30) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compositions and nutritional supplements suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, or a paste. These formulations may be prepared by methods known in the art, e.g., by means of conventional pan-coating, mixing, granulation or lyophilization processes.

Solid dosage forms for oral administration (capsules, tablets, pills, powders, granules and the like) may be prepared by mixing the active ingredient(s) with one or more acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as capsules, pills and granules, may optionally be scored or prepared with coatings and shells, such as enteric coatings and other coatings well known in the art. The tablets, and other solid dosage forms, may also be formulated so as to provide slow or controlled release of the active ingredient therein. They may be sterilized by, for example, filtration through a bacteria-retaining filter. These compositions may also optionally contain opacifying agents that release the active ingredient only, or preferentially, in a certain portion of the gastrointestinal tract, optionally, in a delayed manner. The active ingredient can also be in a microencapsulated form.

Liquid dosage forms for oral administration include acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### EXAMPLES

The following Examples describe various compositions of the present invention, which include butyrate salts (and/or esters or propionate salts thereof), in combination with one or more gallates (namely, epigallocatechin gallate (EGCG)), and, optionally, beta-hydroxybutyrate salts (and/or esters or propionate salts thereof). The invention provides that additional optimizing ingredients may be included in the formulations exemplified below. For example, acetyl carnitine may be included, to provide a fatty acid transport mechanism facilitator. In addition, R-alpha lipoic acid may be included to improve insulin efficacy and to drive serum glucose levels down to be conducive to ketogenesis and ketone body prevalence as an ATP substrate. In the capsule Examples listed below, the capsule containers may consist of vegetable capsules (hydroxypropyl methylcellulose capsules) and, in addition to the active agents listed below, may include magnesium stearate, water, and silicon dioxide.

### Example 1—Capsules (Serving Size=3-4 Capsules)

| Component | Amount/Capsule (mg) |
|---|---|
| Epigallocatechin Gallate | 200 |
| Butyrate Sodium Salt; | 200 |
| Butyrate Calcium Salt; and/or | |
| Butyrate Magnesium Salt | |
| Total Per Capsule(s) | 400 |

### Example 2—Capsules (Serving Size=2 Capsules)

| Component | Amount/Capsule (mg) |
|---|---|
| Epigallocatechin Gallate | 400 |
| Butyrate Sodium Salt; | 100 |
| Butyrate Calcium Salt; and/or | |
| Butyrate Magnesium Salt | |
| Total Per Capsule(s) | 500 |

### Example 3—Capsules (Serving Size=3-4 Capsules)

| Component | Amount/Capsule (mg) |
|---|---|
| Epigallocatechin Gallate | 300 |
| Butyrate Sodium Salt; | 200 |
| Butyrate Calcium Salt; and/or | |
| Butyrate Magnesium Salt | |
| Total Per Capsule(s) | 500 |

### Example 4—Capsules (Serving Size=1-2 Capsules)

| Component | Amount/Capsule (mg) |
|---|---|
| Epigallocatechin Gallate | 220 |
| Butyrate Calcium Salt | 150 |
| Total Per Capsule(s) | 370 |

### Example 5—Capsules (Serving Size=1-2 Capsules)

| Component | Amount/Capsule (mg) |
|---|---|
| Epigallocatechin Gallate | 200 |
| Butyrate Calcium Salt | 160 |
| Total Per Capsule(s) | 360 |

### Example 6—Capsules, Powder, or Liquid Forms

| Component | Amount/Capsule (mg) |
|---|---|
| Epigallocatechin Gallate | 400 |
| Butyrate Calcium Salt | 100 |
| Total Per Capsule(s) | 500 |

### Example 7—Capsules, Powder, or Liquid Forms

| Component | Amount/Capsule (mg) |
|---|---|
| Epigallocatechin Gallate | 400 |
| Butyrate Calcium Salt | 50 |
| Total Per Capsule(s) | 450 |

### Example 8—Capsules, Powder, or Liquid Forms

| Component | Amount/Capsule (mg) |
|---|---|
| Epigallocatechin Gallate | 300 |
| Butyrate Calcium Salt | 180 |
| Total Per Capsule(s) | 480 |

### Example 9—Capsules, Powder, or Liquid Forms

| Component | Amount/Capsule (mg) |
|---|---|
| Epigallocatechin Gallate | 100-500 |
| Butyrate Calcium Salt | 50-250 |
| Total Per Capsule(s) | 150-750 |

### Example 10—Dry Powder

| Component | Powder Serving (mg) |
|---|---|
| Epigallocatechin Gallate | 500 |
| Butyrate Sodium Salt; | 225 |
| Butyrate Calcium Salt; and/or | |
| Butyrate Magnesium Salt | |
| Total Per Dose | 725 |

### Example 11—Dry Powder

| Component | Powder Serving (mg) |
|---|---|
| Epigallocatechin Gallate | 500 |
| Butyrate Sodium Salt; | |
| Butyrate Calcium Salt; and/or | 225 |
| Butyrate Magnesium Salt | |
| Beta Hydroxybutyrate Sodium Salt; | |
| Beta Hydroxybutyrate Calcium Salt; and/or | 3,500 |
| Beta Hydroxybutyrate Magnesium Salt | |
| Total Per Dose | 4,225 |

### Example 12—Effects On Brown Fat Cell Differentiation and Activation

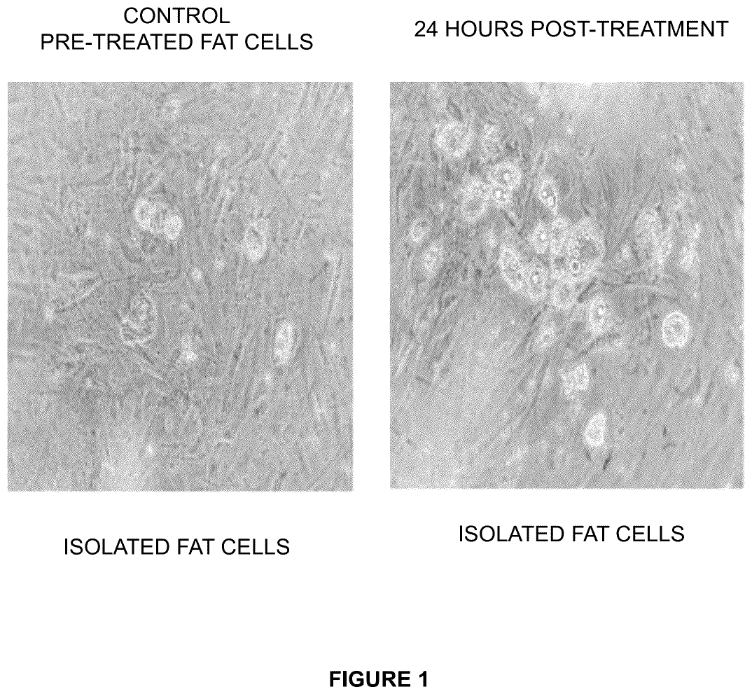

In this Example 12, a composition of the present invention was administered to fibroblast cells that had been differentiated into brown adipocytes. More particularly, a test composition of the present invention was prepared that included a final concentration of 50 μM of EGCG and 2.4 mM of butyrate calcium salt. The differentiated brown adipocytes were incubated in 1,750 μL of the test composition of the present invention at room temperature for a 24-hour period. The differentiated brown adipocytes were viewed and analyzed via standard histology techniques pre- and post-treatment. As shown in the histology images of FIG. 1, after only a 24-hour period, the test composition of the present invention induced significant brown fat cell differentiation and activation.

### Example 13—Effects On Sirtuin-1 Expression

In this Example 13, a composition of the present invention was applied to fibroblast cells that had been differentiated into brown adipocyte-like cells, and the cells were subsequently lysed and the amount of SIRT1 protein in the lysate was subsequently measured. More particularly, the brown adipocyte-like cells were provided to a series of wells in a microtiter plate. A series of test or control compositions were then applied to a duplicate set of wells that contained the cells. Specifically, a test or control composition was applied to each pair of wells containing the cells, with such test and control compositions being cell media alone (RPMI) (control); 120 μL of butyrate calcium salt (1.2 mM); 120 μL of beta-hydroxybutyrate calcium salt (1.2 mM); 10 μL of epigallocatechin gallate (EGCG) (50 μM); 120 μL of butyrate calcium salt (1.2 mM)+120 μL of beta-hydroxybutyrate calcium salt (1.2 mM); or 120 μL of butyrate calcium salt (1.2 mM)+10 μL of epigallocatechin gallate (EGCG) (50 μM).

The cells and test/control compositions were then incubated at 37-degrees Celsius with 5% CO₂ for 48 hours (2 days). The cells were subsequently harvested with a cell scraper and lysed in the presence of a protease inhibitor. An aliquot representing 20 μg of sample from each protein extract was then subjected to acrylamide gel electrophoresis (10% acrylamide gel) for about 50 minutes at 60 mAMP constant and 275 V-hour. The gel was subsequently blotted (subjected to Western Blot techniques) onto a nitrocellulose membrane. The membrane was then blocked in a 2.5% BSA/TTBS solution for 1 hour and subsequently rinsed in TTBS. The membrane was then incubated and probed with a primary rabbit anti-SIRT1 polyclonal antibody (diluted 1:1000) (New England Biosciences) and a rabbit anti-GAPDH polyclonal antibody (diluted 1:1000) at 4-degrees Celsius overnight. The membrane was then rinsed in TTBS and subjected to a secondary antibody incubation (goat anti-rabbit polyclonal antibody at 1:10,000) for 1 hour. The membrane was subsequently subjected to a staining procedure (through an enzyme tethered to the secondary antibody)—and the bands were subsequently visualized and quantified using a Quantity One scanner and densitometry software.

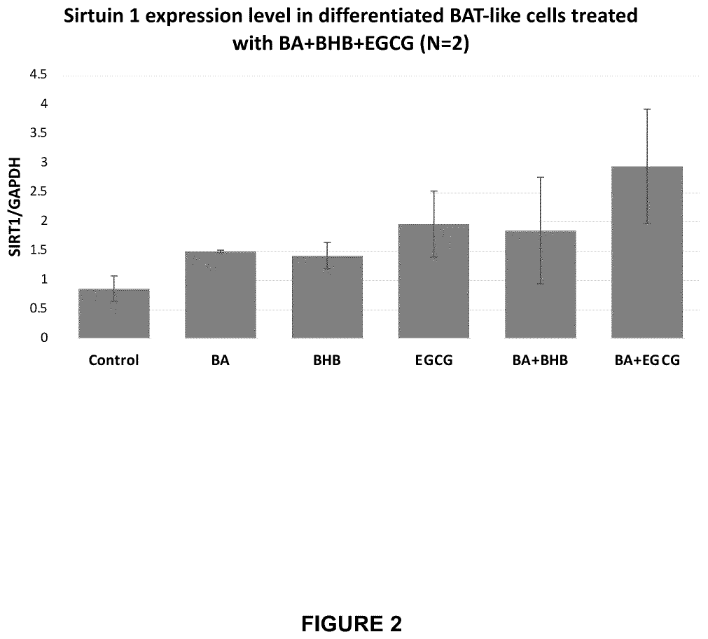

The amount of SIRT1 protein found to be present in each group of wells was normalized to the detected and quantified GAPDH expression. FIG. 2 summarizes the results of this analysis. As shown therein, the cells treated with a combination of butyrate calcium salt (“BA”) and epigallocatechin gallate (“EGCG”) induced expression of SIRT1 significantly more than the control, butyrate calcium salt alone, beta-hydroxybutyrate calcium salt (“BHB”) alone, EGCG alone, and the combination of BA and BHB. As used herein, SIRT1 and Sirtuin-1 refer to NAD-dependent deacetylase sirtuin-1. As this Example 13 demonstrates, the compositions of the present invention, and particularly combinations of butyrate and epigallocatechin gallate, can be used to induce and increase the expression of Sirtuin proteins in cells, and particularly SIRT1.

### Example 14—Effects On Human Weight Loss

In this Example 14, a composition of the present invention was administered to a group of five different human subjects over the course of seven weeks. The dietary habits of the subjects were not altered; however, each subject consumed two capsules by 9:30 am and two capsules at 1:00 pm each day. Each capsule included 225 mg of EGCG and 150 mg of butyrate calcium salt. The subjects also consumed at least 1 liter of water each day. As summarized in FIG. 2, each subject exhibited an impressive amount of weight loss, particularly given that the dietary habits of the subjects were not otherwise altered.

The many aspects and benefits of the invention are apparent from the detailed description, and thus, it is intended for the following claims to cover all such aspects and benefits of the invention that fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A composition comprising:
(a) one or more short chain fatty acids selected from the group consisting of butyric acid, butyrate salts, propionic acid, propionate salts, acetic acid, and acetate salts; and
(b) one or more gallates.

2. The composition of claim 1, wherein the one or more gallates includes epigallocatechin gallate (EGCG).

3. The composition of claim 1, wherein the one or more gallates includes at least 100 mg of epigallocatechin gallate (EGCG).

4. The composition of claim 3, wherein the one or more short chain fatty acids consists of butyrate salts selected from the group consisting of butyrate sodium salt, butyrate calcium salt, and butyrate magnesium salt.

5. The composition of claim 4, wherein the composition is packaged as an oral capsule, which includes at least 200 mg of epigallocatechin gallate (EGCG) and at least 200 mg of purified butyrate salts.

6. The composition of claim 4, wherein the composition is packaged as an oral capsule, which includes at least 400 mg of epigallocatechin gallate (EGCG) and at least 100 mg of purified butyrate salts.

7. The composition of claim 4, wherein the composition is packaged as an oral capsule, which includes at least 300 mg of epigallocatechin gallate (EGCG) and at least 200 mg of purified butyrate salts.

8. The composition of claim 4, wherein the composition is packaged as a dry powder, which includes at least 500 mg of epigallocatechin gallate (EGCG) and at least 225 mg of purified butyrate salts.

9. The composition of claim 4, which further comprises purified beta-hydroxybutyrate or esters or propionate salts thereof, wherein the purified beta-hydroxybutyrate includes a beta-hydroxybutyrate sodium salt, a beta-hydroxybutyrate calcium salt, and/or a beta-hydroxybutyrate magnesium salt.

10. A method for inducing weight loss in a subject, which comprises administering an effective amount of the composition of claim 1 to the subject.

11. A method for inducing brown adipose fat cell differentiation in a subject, which comprises administering an effective amount of the composition of claim 1 to the subject.

12. A method for inducing SIRT1 expression in a group of cells, which comprises administering an effective amount of the composition of claim 1 to the group of cells.
